(19) United States
(12) Patent Application Publication
(10) Pub. No.: US 2026/0228974 A1
(43) Pub. Date: Aug. 6, 2026

(54) METHOD AND SYSTEM FOR ESTABLISHING COLOR DIMENSIONAL MODEL

(71) Applicant: QISDA CORPORATION, Taoyuan City (TW)

(72) Inventors: YAN-HONG CHEN, Taoyuan City (TW); CHING-HUEY WANG, Taoyuan City (TW); TSUNG-HSUN WU, Taoyuan City (TW)

(21) Appl. No.: 19/449,344

(22) Filed: Jan. 14, 2026

(30) Foreign Application Priority Data
Feb. 5, 2025 (CN) 202510129597.8

(51) Int. Cl. G06T 17/10; G06T 7/90

(52) U.S. Cl. CPC G06T 17/10; G06T 7/90

(57) ABSTRACT

A method and system for constructing a color three-dimensional model. The method includes: configuring an image sensing device to capture a first image when a first structured light and a second background light simultaneously illuminate a to-be-measured object, capture a second image when a second structured light and the second background light simultaneously illuminate the to-be-measured object, capture a third image when a first background light illuminates the to-be-measured object, and capture a fourth image when a third background light illuminates the to-be-measured object; executing a decoding process to obtain three-dimensional spatial position information of the to-be-measured object; executing a color mixing process to obtain spatial color information of the to-be-measured object; and generating a target color three-dimensional model of the to-be-measured object based on a spatial correspondence between the three-dimensional spatial position information and the spatial color information.

### CROSS-REFERENCE TO RELATED PATENT APPLICATION

This application claims the benefit of priority to China Patent Application No. 202510129597.8, filed on February 5, 2025, in the People’s Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to the field of intraoral scanners, and more particularly to a method and system for constructing a color three-dimensional model.

### BACKGROUND OF THE DISCLOSURE

An intraoral scanner is an advanced dental device used to rapidly and accurately acquire three-dimensional images of a patient's oral cavity. The intraoral scanner is equipped with a high-precision scanner that employs optical technologies, such as laser or structured light, so as to capture detailed anatomical structures of teeth and gums and generate high-resolution digital models thereof.

In sampling operations utilizing a high-precision scanner, a monochrome sensor is generally employed to prevent degradation of resolution associated with the use of Bayer filters or similar filter structures on the photosensitive element. Specifically, in order to obtain high-resolution images, red, green, and blue light sources are usually used to illuminate the object. A monochrome sensor captures images corresponding to the red, green, and blue colors separately, and these images are subsequently combined to render the object's true color. This method avoids the resolution loss caused by Bayer filters and provides higher-quality images.

The existing intraoral scanners often require multiple sets of structured light and monochromatic light to separately acquire three-dimensional data and color data of an object, and subsequently combine them to reconstruct point cloud data. However, as the number of images used in the reconstruction process increases, the processing time correspondingly lengthens, which adversely affects overall efficiency. Furthermore, variations in lens viewing angles during image capture are more likely to result in degradation of image quality.

### SUMMARY OF THE DISCLOSURE

In response to the above-referenced technical inadequacies, the present disclosure provides a method and system for establishing a color three-dimensional model capable of improving the efficiency and quality of three-dimensional image reconstruction.

To achieve the foregoing purpose, the present disclosure provides a method and a system for establishing a color three-dimensional model. The method for establishing the color three-dimensional model includes: configuring a projection illumination module to illuminate a to-be-measured object with a first structured light, a second structured light, and a first background light of a first wavelength, a second background light of a second wavelength, and a third background light of a third wavelength, in which the first structured light includes a plurality of first structured light patterns, the second structured light includes a plurality of second structured light patterns, and a spatial characteristic of the plurality of second structured light patterns is different from a spatial characteristic of the plurality of first structured light patterns; configuring an image sensing device to capture a first image when the to-be-measured object is simultaneously illuminated by the first structured light and the second background light, to capture a second image when the to-be-measured object is simultaneously illuminated by the second structured light and the second background light, to capture a third image when the to-be-measured object is illuminated by the first background light, and to capture a fourth image when the to-be-measured object is illuminated by the third background light; and configuring a processing device to perform following processes: obtaining the first image, the second image, the third image, and the fourth image; executing a decoding process on the first structured light patterns in the first image and the second structured light patterns in the second image to obtain three-dimensional spatial position information of the to-be-measured object; executing a color mixing process on portions of the first image and the second image corresponding to the second wavelength, a portion of the third image corresponding to the first wavelength, and a portion of the fourth image corresponding to the third wavelength, to obtain spatial color information of the to-be-measured object; and combining the three-dimensional spatial position information and the spatial color information based on a spatial correspondence between the three-dimensional spatial position information and the spatial color information, so as to generate a target color three-dimensional model of the to-be-measured object.

Preferably, when the to-be-measured object is simultaneously illuminated with the first structured light and the second background light, a plurality of first background light patterns are determined based on the plurality of first structured light patterns, and the plurality of first structured light patterns and the plurality of first background light patterns are alternately arranged at a plurality of encoding positions to form a first encoding pattern; when the to-be-measured object is simultaneously illuminated with the second structured light and the second background light, a plurality of second background light patterns are determined based on the plurality of second structured light patterns, and the plurality of second structured light patterns and the plurality of second background light patterns are alternately arranged at the plurality of encoding positions to form a second encoding pattern.

Preferably, the method for establishing the color three-dimensional model includes: configuring a projection illumination module to illuminate a to-be-measured object with a first structured light, a second structured light, a third structured light, a fourth structured light, and a first background light of a first wavelength, a second background light of a second wavelength, and a third background light of a third wavelength, in which the first structured light includes a plurality of first structured light patterns, the second structured light includes a plurality of second structured light patterns, the third structured light includes a plurality of third structured light patterns, the fourth structured light includes a plurality of fourth structured light patterns, and the plurality of first structured light patterns, the plurality of second structured light patterns, the plurality of third structured light patterns, and the plurality of fourth structured light patterns have different spatial characteristics; configuring an image sensing device to capture a first image when the to-be-measured object is simultaneously illuminated by the first structured light and the second background light, to capture a second image when the to-be-measured object is simultaneously illuminated by the second structured light and the second background light, to capture a third image when the to-be-measured object is illuminated by the first background light, to capture a fourth image when the to-be-measured object is simultaneously illuminated by the third structured light and the third background light, and to capture a fifth image when the to-be-measured object is simultaneously illuminated by the fourth structured light and the third background light; executing a decoding process on the plurality of first structured light patterns in the first image, the plurality of second structured light patterns in the second image, the plurality of third structured light patterns in the fourth image, and the plurality of fourth structured light patterns in the fifth image, so as to obtain three-dimensional spatial position information of the to-be-measured object; executing a color mixing process on portions of the first image and the second image corresponding to the second wavelength, a portion of the third image corresponding to the first wavelength, and portions of the fourth image and the fifth image corresponding to the third wavelength, so as to obtain spatial color information of the to-be-measured object; and combining the three-dimensional spatial position information and the spatial color information based on a spatial correspondence between the three-dimensional spatial position information and the spatial color information, so as to generate a target color three-dimensional model of the to-be-measured object.

Preferably, the system for establishing the color three-dimensional model includes: a projection illumination module configured to illuminate a to-be-measured object with a first structured light, a second structured light, and a first background light of a first wavelength, a second background light of a second wavelength, and a third background light of a third wavelength, in which the first structured light includes a plurality of first structured light patterns, the second structured light includes a plurality of second structured light patterns, and a spatial characteristic of the plurality of second structured light patterns is different from a spatial characteristic of the plurality of first structured light patterns; an image sensing device configured to capture a first image when the to-be-measured object is simultaneously illuminated by the first structured light and the second background light, to capture a second image when the to-be-measured object is simultaneously illuminated by the second structured light and the second background light, to capture a third image when the to-be-measured object is illuminated by the first background light, and to capture a fourth image when the to-be-measured object is illuminated by the third background light; and a processing device configured to perform the following processes: obtaining the first image, the second image, the third image, and the fourth image; executing a decoding process on the first structured light patterns in the first image and the second structured light patterns in the second image to obtain three-dimensional spatial position information of the to-be-measured object; executing a color mixing process on portions of the first image and the second image corresponding to the second wavelength, a portion of the third image corresponding to the first wavelength, and a portion of the fourth image corresponding to the third wavelength, to obtain spatial color information of the to-be-measured object; and combining the three-dimensional spatial position information and the spatial color information based on a spatial correspondence between the three-dimensional spatial position information and the spatial color information, so as to generate a target color three-dimensional model of the to-be-measured object.

Preferably, the system for establishing the color three-dimensional model includes: a projection illumination module configured to illuminate a to-be-measured object with a first structured light, a second structured light, a third structured light, a fourth structured light, and a first background light of a first wavelength, a second background light of a second wavelength, and a third background light of a third wavelength, in which the first structured light includes a plurality of first structured light patterns, the second structured light includes a plurality of second structured light patterns, the third structured light includes a plurality of third structured light patterns, the fourth structured light includes a plurality of fourth structured light patterns, and the plurality of first structured light patterns, the plurality of second structured light patterns, the plurality of third structured light patterns, and the plurality of fourth structured light patterns have different spatial characteristics; an image sensing device configured to capture a first image when the to-be-measured object is simultaneously illuminated by the first structured light and the second background light, to capture a second image when the to-be-measured object is simultaneously illuminated by the second structured light and the second background light, to capture a third image when the to-be-measured object is illuminated by the first background light, to capture a fourth image when the to-be-measured object is simultaneously illuminated by the third structured light and the third background light, and to capture a fifth image when the to-be-measured object is simultaneously illuminated by the fourth structured light and the third background light; a processing device configured to perform following processes: obtaining the first image, the second image, the third image, the fourth image, and the fifth image; executing a decoding process on the plurality of first structured light patterns in the first image, the plurality of second structured light patterns in the second image, the plurality of third structured light patterns in the fourth image, and the plurality of fourth structured light patterns in the fifth image, so as to obtain three-dimensional spatial position information of the to-be-measured object; executing a color mixing process on portions of the first image and the second image corresponding to the second wavelength, a portion of the third image corresponding to the first wavelength, and portions of the fourth image and the fifth image corresponding to the third wavelength, so as to obtain spatial color information of the to-be-measured object; and combining the three-dimensional spatial position information and the spatial color information based on a spatial correspondence between the three-dimensional spatial position information and the spatial color information, so as to generate a target color three-dimensional model of the to-be-measured object.

Therefore, the method and system for establishing the color three-dimensional model provided by the present disclosure can, when the object is simultaneously illuminated with the background light and the structured light, form an encoding pattern with a specially designed structure. During image capture, the structured light can be clearly identified, and all encoding positions contain complete color information corresponding to a specific wavelength. Moreover, a quantity of images required for reconstructing the color three-dimensional model can be significantly reduced. As a result, the efficiency of color three-dimensional model reconstruction can be improved while maintaining image quality.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a functional block diagram of a system for establishing a color three-dimensional model according to one embodiment of the present disclosure;

FIG. 2 is a schematic diagram illustrating a configuration of a projection illumination module and an image sensing device according to the present disclosure;

FIG. 3 is a flowchart of the method for establishing a color three-dimensional model according to one embodiment of the present disclosure;

FIG. 4 is a schematic diagram illustrating a configuration of structured lights and background lights according to one embodiment of the present disclosure;

FIG. 5 is a schematic diagram illustrating an image capturing configuration under simultaneous illumination of the structured light and the background light according to one embodiment of the present disclosure;

FIG. 6 is another schematic diagram illustrating the configuration of the structured light and the background light according to one embodiment of the present disclosure;

FIG. 7 is another schematic diagram illustrating the image capturing configuration under simultaneous illumination of the structured light and the background light according to one embodiment of the present disclosure;

FIGS. 8 and 9 are images captured when the to-be-measured object is simultaneously illuminated with a red background light and two different configurations of structured lights; and

FIG. 10 is a schematic diagram illustrating a color mixing process according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

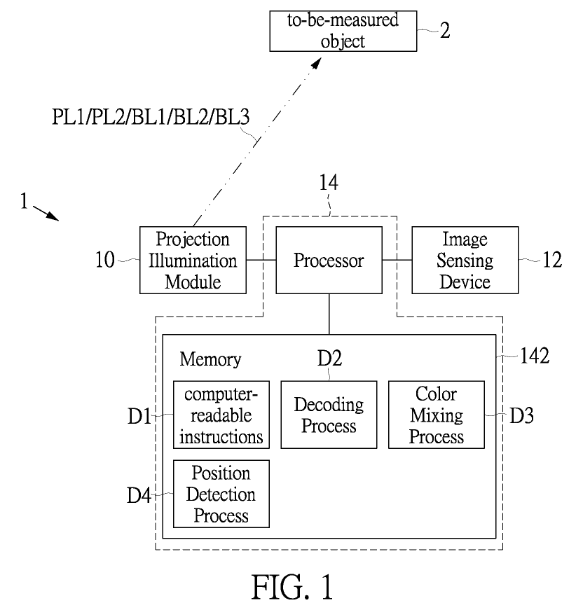

FIG. 1 is a functional block diagram of the system for establishing a color three-dimensional model according to one embodiment of the present disclosure. Referring to FIG. 1, a first embodiment of the present disclosure provides a system 1 for constructing a color three-dimensional model, the system 1 includes a projection illumination module 10, an image sensing device 12, and a processing device 14.

In this embodiment, the projection illumination module 10 can be configured to generate a first structured light PL1, a second structured light PL2, a first background light BL1, a second background light BL2, and a third background light BL3. The first structured light PL1, the second structured light PL2, and the first background light BL1 can have a first wavelength, the second background light BL2 can have a second wavelength, and the third background light BL3 can have a third wavelength, and the first wavelength, the second wavelength, and the third wavelength are different from each other.

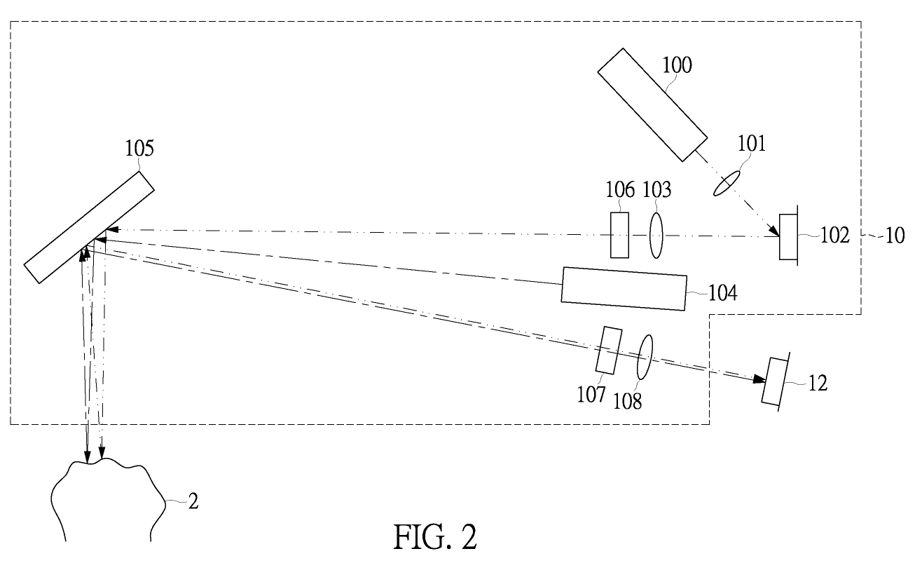

FIG. 2 is a schematic diagram illustrating a configuration of a projection illumination module and an image sensing device according to the present disclosure. Referring to FIG. 2, the projection illumination module 10 can include a first light source 100, a projection pattern generator 102, and a second light source 104. The first light source 100 and the second light source 104 can, for example, be light-emitting diodes (LEDs) or laser diodes, and the projection pattern generator 102 can, for example, be a digital micromirror device (DMD) or a liquid crystal display (LCD). The first light source 100 and the second light source 104 can project light in the same or different wavelength bands. In a specific embodiment, the first light source 100 can project structured light at designated wavelengths and structures through the projection pattern generator 102, such as the first structured light PL1 and the second structured light PL2 of the first wavelength. The second light source 104 can project background light at designated wavelengths, such as the first background light BL1 of the first wavelength, the second background light BL2 of the second wavelength, and the third background light BL3 of the third wavelength.

In the projection illumination module 10, one or more optical elements can be provided, including, for example, focusing lenses 101, 103, and 108, collimating lenses 106 and 107, and a reflecting mirror 105, for focusing and collimating the first structured light PL1, the second structured light PL2, the first background light BL1, the second background light BL2, and the third background light BL3 and guiding them to the to-be-measured object 2, as well as for focusing and collimating the light reflected from the to-be-measured object 2 and guiding it to the image sensing device 12 for image capturing. It should be noted that the optical configuration adopted by the projection illumination module 10 is not limited to that shown in FIG. 2 and can be adjusted based on requirements, including a quantity and types of light sources, projection pattern generators, and optical elements. Further, the collimating lenses 106 and 107 can include fly-eye lenses; however, the present disclosure is not limited thereto.

The image sensing device 12 can, for example, be a complementary metal-oxide-semiconductor (CMOS) sensor or a charge-coupled device (CCD) sensor. Both sensor technologies have respective advantages and are widely used in digital cameras and imaging devices to capture optical signals and convert them into electrical signals. For example, CMOS sensors offer advantages such as low power consumption and high-speed readout, while CCD sensors offer high sensitivity and low noise characteristics.

The processing device 14 can be electrically connected to the projection illumination module 10 and the image sensing device 12, and is responsible for coordinating the operation of the light sources (such as the first light source 100 and the second light source 104), the projection pattern generator 102, and the image sensing device 12, as well as processing the image data captured by the image sensing device 12. In some embodiments, the processing device 14 can include a processor 140 and a memory 142. The memory 142 can store a plurality of computer-readable instructions D1, which are read by the processor 140 to perform image processing and three-dimensional reconstruction procedures, such as including a decoding process D2, a color mixing process D3, and a position detection process D4.

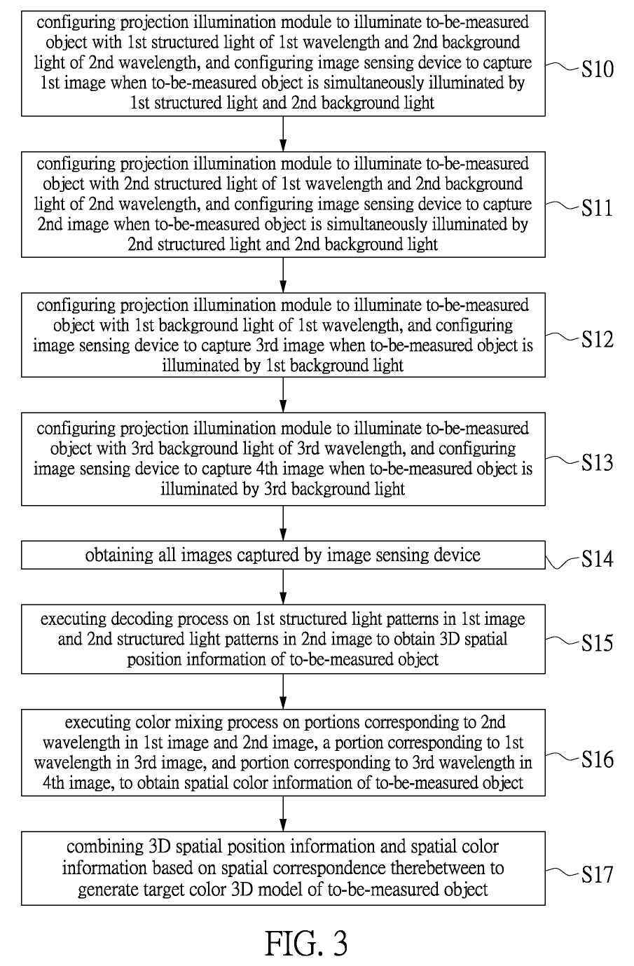

Referring to FIG. 3, FIG. 3 is a flowchart of the method for establishing a color three-dimensional model according to one embodiment of the present disclosure. As shown in FIG. 3, one embodiment of the present disclosure provides a method for establishing a color three-dimensional model, which is applicable to the system 1 for establishing the color three-dimensional model shown in FIG. 1, and includes at least the following steps:

Step S10: configuring the projection illumination module to illuminate the to-be-measured object with first structured light of the first wavelength and the second background light of the second wavelength, and configuring the image sensing device to capture a first image when the to-be-measured object is simultaneously illuminated by the first structured light and the second background light.

Step S11: configuring the projection illumination module to illuminate the to-be-measured object with second structured light of the first wavelength and the second background light of the second wavelength, and configuring the image sensing device to capture a second image when the to-be-measured object is simultaneously illuminated by the second structured light and the second background light.

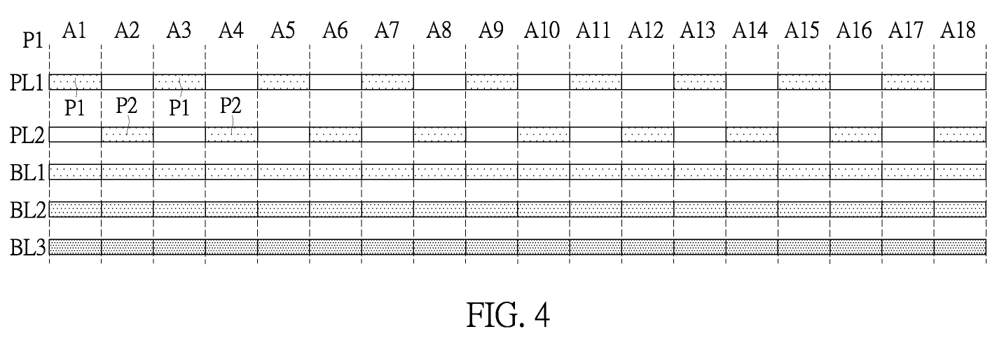

It should be noted that, in the above steps, the first structured light PL1 and the second structured light PL2 can have different spatial characteristics. Referring to FIG. 4, FIG. 4 is a schematic diagram illustrating a configuration of structured lights and background lights according to one embodiment of the present disclosure. As shown in FIG. 4, the first structured light PL1 includes a plurality of first structured light patterns P1, and the second structured light PL2 includes a plurality of second structured light patterns P2. The plurality of first structured light patterns P1 and the plurality of second structured light patterns P2 can, for example, be a plurality of line patterns, rectangular patterns, or other geometric patterns arranged in parallel along a certain direction. The first structured light patterns P1 can be arranged in a manner different from that of the second structured light patterns P2, thereby exhibiting different spatial characteristics.

For example, the arrangement of the first structured light patterns P1 and the second structured light patterns P2 can be determined based on a predetermined encoding configuration. In the predetermined encoding configuration, a plurality of encoding positions A1 to A18 are provided. The plurality of first structured light patterns P1 can first be intermittently arranged at the encoding positions A1 to A18, and the plurality of second structured light patterns P2 can then be arranged at the encoding positions not occupied by the first structured light patterns P1 in a complementary manner. For instance, the first structured light patterns P1 can be arranged at a plurality of odd-numbered positions among the encoding positions, that is, at encoding positions A1, A3, ..., and A17, while the second structured light patterns P2 can be arranged at even-numbered positions, that is, at encoding positions A2, A4, ..., and A18. In addition, the first background light BL1 and the second background light BL2 are not encoded and thus appear at all the encoding positions A1 to A18.

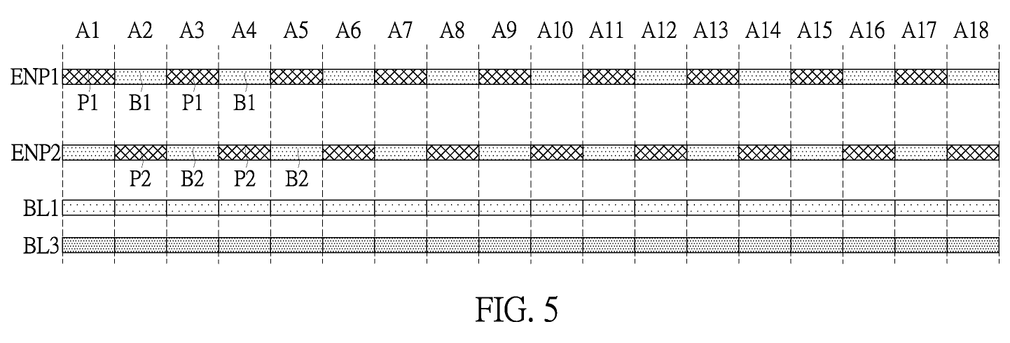

Referring to FIG. 5, FIG. 5 is a schematic diagram illustrating an image capturing configuration under simultaneous illumination of the structured light and the background light according to one embodiment of the present disclosure. When the to-be-measured object is simultaneously illuminated with the first structured light PL1 and the second background light BL2, a plurality of first background light patterns B1 are determined based on the plurality of first structured light patterns P1, and the plurality of first structured light patterns P1 and the plurality of first background light patterns B1 are alternately arranged at the encoding positions A1 to A18 to form a first encoding pattern ENP1. Similarly, when the to-be-measured object is simultaneously illuminated with the second structured light PL2 and the second background light BL2, a plurality of second background light patterns B2 are determined based on the plurality of second structured light patterns P2, and the plurality of second structured light patterns P2 and the plurality of second background light patterns B2 are alternately arranged at the encoding positions A1 to A18 to form a second encoding pattern ENP2. Since the second structured light patterns P2 are arranged in a complementary manner relative to the first structured light patterns P1, the encoding positions where the first background light patterns B1 are located are complementary to the encoding positions where the second background light patterns B2 are located.

For example, the plurality of first background light patterns B1 are respectively disposed at the even-numbered positions among the encoding positions, such as the encoding positions A2, A4, ..., and A18, and the plurality of second background light patterns B2 are respectively disposed at the odd-numbered positions, such as the encoding positions A1, A3, ..., and A17. Accordingly, the encoding positions A1, A3, ..., and A17 in the original first structured light PL1, where the first structured light patterns P1 are arranged, cannot provide accurate color information. By simultaneously projecting the second background light BL2 and the second structured light PL2 having encoding positions different from those of the first structured light PL1, the encoding positions A1, A3, ..., and A17 can be utilized to provide color information. Similarly, the encoding positions A2, A4, ..., and A18 in the original second structured light PL2, where the second structured light patterns P2 are arranged, cannot provide accurate color information. By simultaneously projecting the second background light BL2 and the first structured light PL1 having encoding positions different from those of the second structured light PL2, the encoding positions A2, A4, ..., and A18 can be utilized to provide accurate color information.

In addition, when the to-be-measured object is simultaneously illuminated with the first structured light PL1 and the second background light BL2, and when the to-be-measured object is simultaneously illuminated with the second structured light PL2 and the second background light BL2, the first structured light patterns P1 and the second structured light patterns P2 formed on the to-be-measured object have a fourth wavelength that is different from the first wavelength, the second wavelength, and the third wavelength. For example, the first wavelength, the second wavelength, and the third wavelength can respectively correspond to blue light, red light, and green light, such that the fourth wavelength, generated by simultaneously projecting blue light and red light, corresponds to magenta light. Therefore, in the first image and the second image captured in steps S10 and S11, positions of the first structured light patterns P1 and the second structured light patterns P2 can still be clearly identified and can be used for subsequent decoding to obtain position information.

Referring again to FIG. 3, the method for establishing the color three-dimensional model proceeds to step S12: configuring the projection illumination module to illuminate the to-be-measured object with the first background light of the first wavelength, and configuring the image sensing device to capture a third image when the to-be-measured object is illuminated by the first background light.

Step S13: configuring the projection illumination module to illuminate the to-be-measured object with the third background light of the third wavelength, and configuring the image sensing device to capture a fourth image when the to-be-measured object is illuminated by the third background light. Referring to FIGS. 4 and 5, since the first background light BL1 and the third background light BL3 are not encoded, they appear at all the encoding positions A1 to A18 when capturing the third image and the fourth image.

In the above steps, the projection illumination module 10 and the image sensing device 12 can perform high-speed synchronized projection and image capture, and the capture speed can be greater than or equal to 150 frames per second (fps). In addition, the configuration of the structured light and the background light adopted in the present disclosure is not limited thereto.

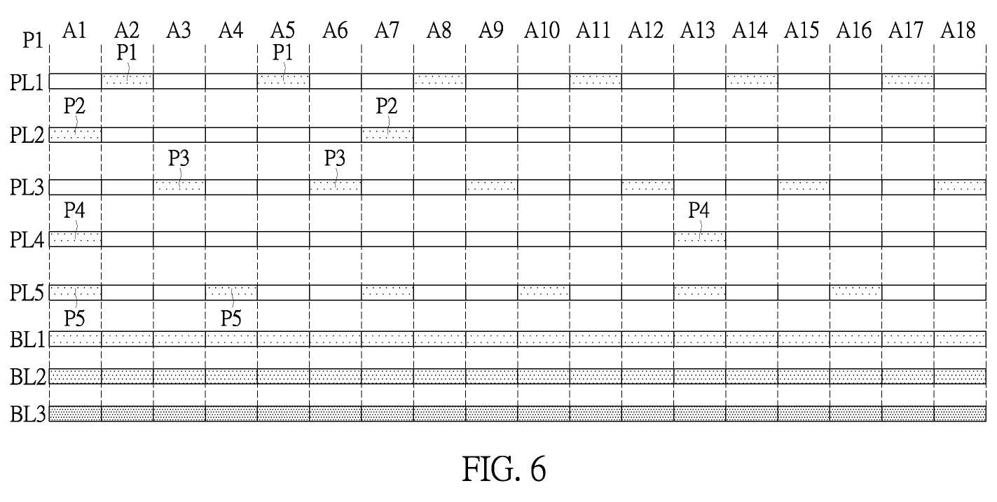

Referring to FIG. 6, FIG. 6 is another schematic diagram illustrating the configuration of the structured lights and the background lights according to one embodiment of the present disclosure. In addition to the first structured light PL1 and the second structured light PL2, the projection illumination module 10 can also be used to illuminate the to-be-measured object 2 with a third structured light PL3, a fourth structured light PL4, and a fifth structured light PL5 of the first wavelength.

In this embodiment, another predetermined encoding configuration can be used to determine the arrangement of the structured light patterns in the first structured light PL1, the second structured light PL2, the third structured light PL3, the fourth structured light PL4, and the fifth structured light PL5. In the another predetermined encoding configuration, a plurality of encoding positions A1 to A18 are provided. The first structured light PL1 includes a plurality of first structured light patterns P1 disposed at encoding positions A2, A5, A8, A11, A14, and A17. The second structured light PL2 includes two second structured light patterns P2 disposed at encoding positions A1 and A4. The third structured light PL3 includes a plurality of third structured light patterns P3 disposed at encoding positions A3, A6, A9, A12, A15, and A18. The fourth structured light PL4 includes two fourth structured light patterns P4 disposed at encoding positions A1 and A13. The fifth structured light PL5 includes a plurality of fifth structured light patterns P5 disposed at encoding positions A1, A4, A7, A10, A13, and A16. Thus, the above first structured light patterns P1, second structured light patterns P2, third structured light patterns P3, fourth structured light patterns P4, and fifth structured light patterns P5 exhibit different spatial characteristics.

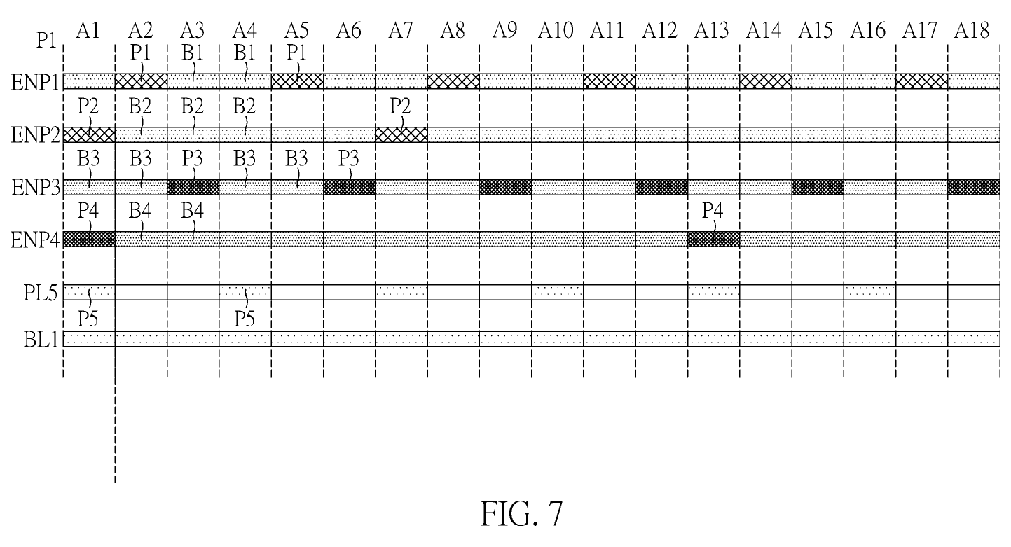

Under this structured light configuration, a quantity of images to be captured will also change accordingly. Referring to FIG. 7, FIG. 7 is another schematic diagram illustrating the image capturing configuration under simultaneous illumination of the structured light and the background light according to one embodiment of the present disclosure. Similarly, by executing Step S10 and Step S11, a first image can be captured when the to-be-measured object 2 is simultaneously illuminated by the first structured light PL1 and the second background light BL2, and a second image can be captured when the to-be-measured object 2 is simultaneously illuminated by the second structured light PL2 and the second background light BL2. When the to-be-measured object 2 is simultaneously illuminated with the first structured light PL1 and the second background light BL2, the plurality of first structured light patterns P1 and the plurality of first background light patterns B1 are alternately arranged at the encoding positions A1 to A18 to form a first encoding pattern ENP1, and the plurality of first background light patterns B1 are disposed at encoding positions other than A2, A5, A8, A11, A14, and A17. When the to-be-measured object 2 is simultaneously illuminated with the second structured light PL2 and the second background light BL2, the plurality of second structured light patterns P2 and the plurality of second background light patterns B2 are alternately arranged at the encoding positions A1 to A18 to form a second encoding pattern ENP2, and the plurality of second background light patterns B2 are disposed at encoding positions other than A1 and A4.

Similarly, by executing Step S12, a third image can be captured when the to-be-measured object 2 is illuminated by the first background light BL1. On the other hand, in Step S13, a fourth image is further captured when the to-be-measured object 2 is simultaneously illuminated by the third structured light PL3 and the third background light BL3. When the to-be-measured object 2 is simultaneously illuminated with the third structured light PL3 and the third background light BL3, a plurality of third background light patterns B3 are determined based on the plurality of third structured light patterns P3. The plurality of third structured light patterns P3 and the plurality of third background light patterns B3 are alternately arranged at the encoding positions A1 to A18 to form a third encoding pattern ENP3, and the plurality of third background light patterns B3 are disposed at encoding positions other than A3, A6, A9, A12, A15, and A18.

In addition to the above-mentioned first to fourth images, a fifth image is captured when the to-be-measured object 2 is simultaneously illuminated by the fourth structured light PL4 and the third background light BL3. At this time, a plurality of fourth background light patterns B4 are determined based on the plurality of fourth structured light patterns P4. The plurality of fourth structured light patterns P4 and the plurality of fourth background light patterns B4 are alternately arranged at the encoding positions A1 to A18 to form a fourth encoding pattern ENP4, and the plurality of fourth background light patterns B4 are disposed at encoding positions other than A1 and A13. On the other hand, a sixth image is captured when the to-be-measured object 2 is illuminated by the fifth structured light PL5.

In addition, the first structured light patterns P1 and the second structured light patterns P2 formed on the to-be-measured object 2 have a fourth wavelength different from the first wavelength, the second wavelength, and the third wavelength, while the third structured light patterns P3 and the fourth structured light patterns P4 formed on the to-be-measured object 2 have a fifth wavelength different from the first wavelength, the second wavelength, the third wavelength, and the fourth wavelength. For example, the first wavelength, the second wavelength, and the third wavelength can respectively correspond to blue light, red light, and green light. Accordingly, the fourth wavelength is generated by simultaneously projecting the blue light and the red light, resulting in magenta light, and the fifth wavelength is generated by simultaneously projecting the blue light and the green light, resulting in cyan light.

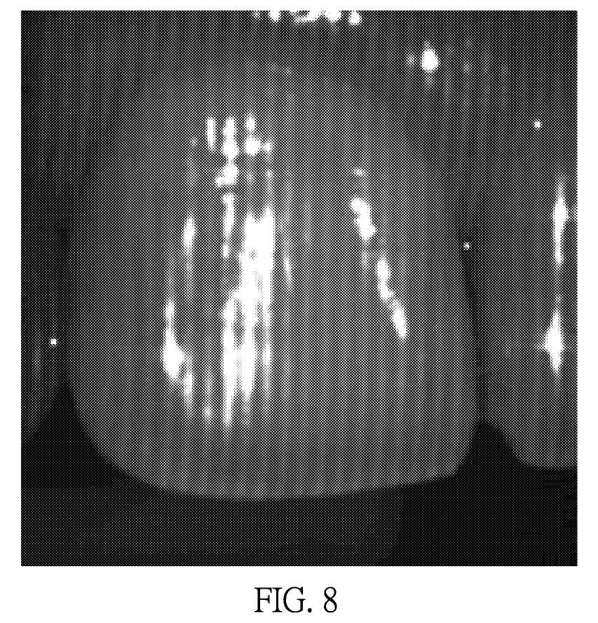

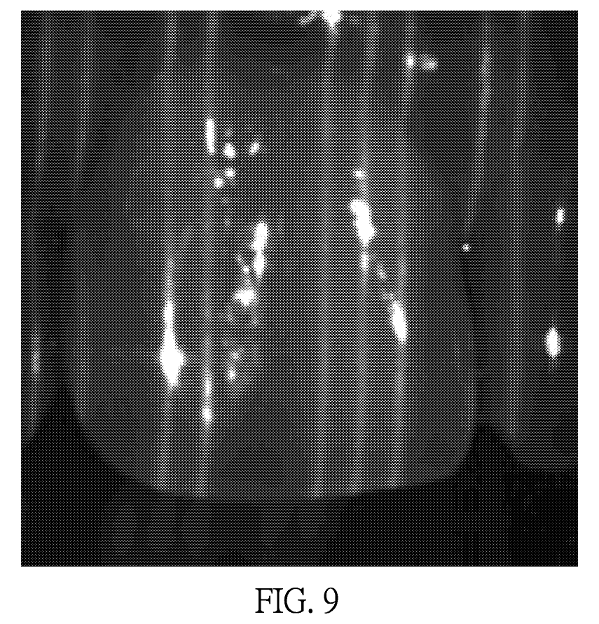

Thus, reference is made to FIGS. 8 and 9, which are images captured when the to-be-measured object is simultaneously illuminated with a red background light and two different configurations of structured lights. As can be seen from FIGS. 8 and 9, the positions of the structured light patterns can still be clearly identified, which can be used for subsequent decoding to obtain position information.

It should be noted that, in the first image and the second image, the portions corresponding to the second wavelength (i.e., the portions occupied by the background light patterns in the first encoding pattern ENP1 and the second encoding pattern ENP2) can at least fill all the encoding positions A1 to A18. Similarly, in the fourth image and the fifth image, the portions corresponding to the third wavelength (i.e., the portions occupied by the background light patterns in the third encoding pattern ENP3 and the fourth encoding pattern ENP4) can also at least fill all the encoding positions A1 to A18. Therefore, it is not necessary to capture images by separately illuminating the to-be-measured object 2 with only the second background light BL2 or the third background light BL3. Complete color information corresponding to the second wavelength and the third wavelength can still be ensured at all encoding positions A1 to A18. As a result, a quantity of required images can be reduced, thereby improving scanning speed and reducing errors.

Referring again to FIG. 3, the method for establishing the color three-dimensional model further includes configuring the processing device 14 to perform the following steps:

Step S14: obtaining all images captured by the image sensing device.

Step S15: executing a decoding process on the plurality of first structured light patterns in the first image and the plurality of second structured light patterns in the second image to obtain three-dimensional spatial position information of the to-be-measured object.

In this step, a position detection process D4 can first be performed on the first image and the second image. For example, a UV detector can be used to determine a centroid position of each structured light pattern (for instance, (Ux, Vx)). A localizer can then locate and encode each structured light pattern based on the centroid position to obtain information to be decoded (for example, (Ui, Vi)). Subsequently, corrections can be made based on pose parameters of the image sensing device (e.g., cameras), and the decoding process D2 is executed to perform decoding, ultimately obtaining pattern position information. For instance, first pattern position information corresponding to the first structured light patterns P1 in the first image, and second pattern position information corresponding to the second structured light patterns P2 in the second image, can be obtained.

As the quantity of captured images increases, pattern position information corresponding to different structured lights can be obtained in a similar manner. For example, as described in descriptions associated with FIG. 7, the first image capturing the first encoding pattern ENP1, the second image capturing the second encoding pattern ENP2, the fourth image capturing the third encoding pattern ENP3, the fifth image capturing the fourth encoding pattern ENP4, and the sixth image capturing the fifth structured light PL5 can each be processed similarly to obtain pattern position information corresponding to each structured light pattern, thereby obtaining complete three-dimensional spatial position information of the to-be-measured object 2.

Step S16: executing a color mixing process on portions corresponding to the second wavelength in the first image and the second image, a portion corresponding to the first wavelength in the third image, and a portion corresponding to the third wavelength in the fourth image, to obtain spatial color information of the to-be-measured object.

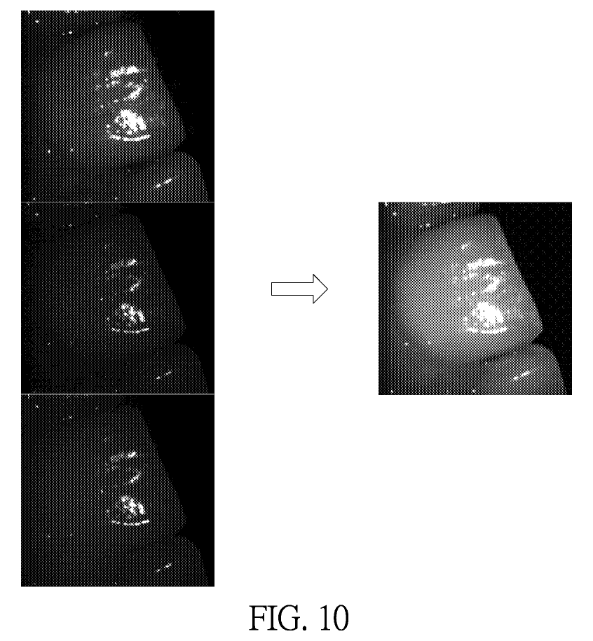

In this step, referring to FIG. 5, since the portions corresponding to the second wavelength in the first image and the second image (i.e., the portions occupied by the background light patterns in the first encoding pattern ENP1 and the second encoding pattern ENP2) can at least fill all the encoding positions A1 to A18, it can be ensured that complete color information corresponding to the second wavelength is available at all encoding positions A1 to A18. Accordingly, by combining the color information corresponding to the second wavelength in the first image and the second image, the color information corresponding to the first wavelength in the third image, and the color information corresponding to the third wavelength in the fourth image, a color image can be obtained by executing the color mixing process D3. Referring to FIG. 10, FIG. 10 is a schematic diagram illustrating a color mixing process according to one embodiment of the present disclosure. The three images on the left side of FIG. 10 respectively represent the color information corresponding to red, green, and blue. After performing the color mixing process, a color image is obtained, as shown on the right side of FIG. 10.

Referring to FIG. 7, since the portions corresponding to the second wavelength in the first image and the second image (i.e., the portions occupied by the background light patterns in the first encoding pattern ENP1 and the second encoding pattern ENP2) can at least fill all the encoding positions A1 to A18, and the portions corresponding to the third wavelength in the fourth image and the fifth image (i.e., the portions occupied by the background light patterns in the third encoding pattern ENP3 and the fourth encoding pattern ENP4) can also at least fill all the encoding positions A1 to A18, it can be ensured that complete color information corresponding to the second wavelength and the third wavelength is available at all encoding positions A1 to A18. Accordingly, by combining the color information corresponding to the second wavelength in the first image and the second image, the color information corresponding to the first wavelength in the third image, and the color information corresponding to the third wavelength in the fourth image and the fifth image, a color image can be obtained by executing the color mixing process D3.

Step S17: combining the three-dimensional spatial position information and the spatial color information based on a spatial correspondence therebetween to generate a target color three-dimensional model of the to-be-measured object.

In this step, referring to FIG. 5, a surface morphology of the to-be-measured object 2 in space can be obtained based on the first pattern position information corresponding to the first structured light patterns P1 in the first image, and the second pattern position information corresponding to the second structured light patterns P2 in the second image. Furthermore, since the portions corresponding to the second wavelength in the first image and the second image, the portion corresponding to the first wavelength in the third image, and the portion corresponding to the third wavelength in the fourth image all have corresponding color position information, and since this color position information is correlated with the first pattern position information and the second pattern position information, the spatial correspondence between the three-dimensional spatial position information and the spatial color information can be further obtained based on such correlation.

Similarly, referring to FIG. 7, the surface morphology of the to-be-measured object 2 in space can be obtained based on the first pattern position information corresponding to the first structured light patterns P1 in the first image, the second pattern position information corresponding to the second structured light patterns P2 in the second image, the third pattern position information corresponding to the third structured light patterns P3 in the fourth image, the fourth pattern position information corresponding to the fourth structured light patterns P4 in the fifth image, and the fifth pattern position information corresponding to the fifth structured light patterns P5 in the sixth image. Furthermore, since the portions corresponding to the second wavelength in the first image and the second image, the portions corresponding to the third wavelength in the fourth image and the fifth image, and the portion corresponding to the first wavelength in the third image all have corresponding color position information, and since all of this color position information is correlated with the respective pattern position information, the spatial correspondence relationship between the three-dimensional spatial position information and the spatial color information can be further obtained based on such correlation.

In step S17, the so-called target color three-dimensional model can include a plurality of model data points (also referred to as point clouds) used to describe the surface morphology of the to-be-measured object 2 in a three-dimensional coordinate system, as well as a plurality of records of color information corresponding to the plurality of model data points. Accordingly, when the method and system for establishing the color three-dimensional model of the present disclosure are applied to a scanner or related fields, the quantity of images required for reconstructing the color three-dimensional model can be significantly reduced. More specifically, the quantity of required images can be fewer than the total number of structured lights and background lights of various wavelengths.

### Beneficial Effects of the Embodiments

In conclusion, the method and system for establishing the color three-dimensional model provided by the present disclosure can, when the object is simultaneously illuminated with the background light and the structured light, form an encoding pattern with a specially designed structure. During image capture, the structured light can be clearly identified, and all encoding positions contain complete color information corresponding to a specific wavelength. Moreover, a quantity of images required for reconstructing the color three-dimensional model can be significantly reduced. As a result, the efficiency of color three-dimensional model reconstruction can be improved while maintaining image quality.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for establishing a color three-dimensional model, the method comprising:
configuring a projection illumination module to illuminate a to-be-measured object with a first structured light, a second structured light, and a first background light of a first wavelength, a second background light of a second wavelength, and a third background light of a third wavelength, wherein the first structured light includes a plurality of first structured light patterns, the second structured light includes a plurality of second structured light patterns, and a spatial characteristic of the plurality of second structured light patterns is different from a spatial characteristic of the plurality of first structured light patterns;
configuring an image sensing device to capture a first image when the to-be-measured object is simultaneously illuminated by the first structured light and the second background light, capture a second image when the to-be-measured object is simultaneously illuminated by the second structured light and the second background light, capture a third image when the to-be-measured object is illuminated by the first background light, and capture a fourth image when the to-be-measured object is illuminated by the third background light; and
configuring a processing device to perform following processes:
obtaining the first image, the second image, the third image, and the fourth image;
executing a decoding process on the first structured light patterns in the first image and the second structured light patterns in the second image to obtain three-dimensional spatial position information of the to-be-measured object;
executing a color mixing process on portions of the first image and the second image corresponding to the second wavelength, a portion of the third image corresponding to the first wavelength, and a portion of the fourth image corresponding to the third wavelength, to obtain spatial color information of the to-be-measured object; and
combining the three-dimensional spatial position information and the spatial color information based on a spatial correspondence between the three-dimensional spatial position information and the spatial color information, so as to generate a target color three-dimensional model of the to-be-measured object.

2. The method according to claim 1, further comprising: configuring the processing device to perform following processes:
when the to-be-measured object is simultaneously illuminated with the first structured light and the second background light, determining a plurality of first background light patterns based on the plurality of first structured light patterns, wherein the plurality of first structured light patterns and the plurality of first background light patterns are alternately arranged at a plurality of encoding positions to form a first encoding pattern; and
when the to-be-measured object is simultaneously illuminated with the second structured light and the second background light, determining a plurality of second background light patterns based on the plurality of second structured light patterns, wherein the plurality of second structured light patterns and the plurality of second background light patterns are alternately arranged at the plurality of encoding positions to form a second encoding pattern.

3. The method according to claim 2, wherein the encoding positions of the plurality of first background light patterns are complementary to the encoding positions of the plurality of second background light patterns.

4. The method according to claim 2, wherein: the plurality of first structured light patterns are respectively disposed at a plurality of odd-numbered positions among the plurality of encoding positions, and the plurality of first background light patterns are respectively disposed at a plurality of even-numbered positions among the plurality of encoding positions; and
the plurality of second structured light patterns are respectively disposed at the plurality of even-numbered positions, and the plurality of second background light patterns are respectively disposed at the plurality of odd-numbered positions.

5. The method according to claim 4, wherein, when the to-be-measured object is simultaneously illuminated with the first structured light and the second background light, and when the to-be-measured object is simultaneously illuminated with the second structured light and the second background light, the plurality of first structured light patterns and the plurality of second structured light patterns formed on the to-be-measured object have a fourth wavelength different from the first wavelength, the second wavelength, and the third wavelength.

6. A method for establishing a color three-dimensional model, the method comprising:
configuring a projection illumination module to illuminate a to-be-measured object with a first structured light, a second structured light, a third structured light, a fourth structured light, and a first background light of a first wavelength, a second background light of a second wavelength, and a third background light of a third wavelength, wherein the first structured light includes a plurality of first structured light patterns, the second structured light includes a plurality of second structured light patterns, the third structured light includes a plurality of third structured light patterns, the fourth structured light includes a plurality of fourth structured light patterns, and the plurality of first structured light patterns, the plurality of second structured light patterns, the plurality of third structured light patterns, and the plurality of fourth structured light patterns have different spatial characteristics;
configuring an image sensing device to capture a first image when the to-be-measured object is simultaneously illuminated by the first structured light and the second background light, capture a second image when the to-be-measured object is simultaneously illuminated by the second structured light and the second background light, capture a third image when the to-be-measured object is illuminated by the first background light, capture a fourth image when the to-be-measured object is simultaneously illuminated by the third structured light and the third background light, and capture a fifth image when the to-be-measured object is simultaneously illuminated by the fourth structured light and the third background light;
configuring a processing device to perform following processes:
executing a decoding process on the plurality of first structured light patterns in the first image, the plurality of second structured light patterns in the second image, the plurality of third structured light patterns in the fourth image, and the plurality of fourth structured light patterns in the fifth image, so as to obtain three-dimensional spatial position information of the to-be-measured object;
executing a color mixing process on portions of the first image and the second image corresponding to the second wavelength, a portion of the third image corresponding to the first wavelength, and portions of the fourth image and the fifth image corresponding to the third wavelength, so as to obtain spatial color information of the to-be-measured object; and
combining the three-dimensional spatial position information and the spatial color information based on a spatial correspondence between the three-dimensional spatial position information and the spatial color information, so as to generate a target color three-dimensional model of the to-be-measured object.

7. The method according to claim 1, further comprising:
configuring the projection illumination module to illuminate the to-be-measured object with a fifth structured light of the first wavelength; and
configuring the image sensing device to capture a sixth image when the to-be-measured object is illuminated by the fifth structured light.

8. The method according to claim 7, wherein the process of obtaining the three-dimensional spatial position information of the to-be-measured object further includes: executing the decoding process on a plurality of fifth structured light patterns of the fifth structured light.

9. The method according to claim 6, further comprising: configuring the processing device to perform following processes:
when the to-be-measured object is simultaneously illuminated with the third structured light and the third background light, determining a plurality of third background light patterns based on the plurality of third structured light patterns, wherein the plurality of third structured light patterns and the plurality of third background light patterns are alternately arranged at a plurality of encoding positions to form a third encoding pattern; and
when the to-be-measured object is simultaneously illuminated with the fourth structured light and the third background light, determining a plurality of fourth background light patterns based on the plurality of fourth structured light patterns, wherein the plurality of fourth structured light patterns and the plurality of fourth background light patterns are alternately arranged at the plurality of encoding positions to form a fourth encoding pattern.

10. The method according to claim 9, wherein portions corresponding to the second wavelength in the first image and the second image fill all of the plurality of encoding positions, and portions corresponding to the third wavelength in the fourth image and the fifth image also fill all of the plurality of encoding positions.

11. The method according to claim 1, further comprising: configuring the processing device to perform the following processes:
executing a position detection process on the first image and the second image to obtain first pattern position information of the plurality of first structured light patterns in the first image and second pattern position information of the plurality of second structured light patterns in the second image; and
obtaining, according to the first pattern position information and the second pattern position information, color position information of the portions corresponding to the second wavelength in the first image and the second image, the portion corresponding to the first wavelength in the third image, and the portion corresponding to the third wavelength in the fourth image, and obtaining the spatial correspondence between the three-dimensional spatial position information and the spatial color information from the color position information.

12. A system for establishing a color three-dimensional model, the method comprising:
a projection illumination module configured to illuminate a to-be-measured object with a first structured light, a second structured light, and a first background light of a first wavelength, a second background light of a second wavelength, and a third background light of a third wavelength, wherein the first structured light includes a plurality of first structured light patterns, the second structured light includes a plurality of second structured light patterns, and a spatial characteristic of the plurality of second structured light patterns is different from a spatial characteristic of the plurality of first structured light patterns;
an image sensing device configured to capture a first image when the to-be-measured object is simultaneously illuminated by the first structured light and the second background light, capture a second image when the to-be-measured object is simultaneously illuminated by the second structured light and the second background light, capture a third image when the to-be-measured object is illuminated by the first background light, and capture a fourth image when the to-be-measured object is illuminated by the third background light; and
a processing device configured to perform the following processes:
obtaining the first image, the second image, the third image, and the fourth image;
executing a decoding process on the first structured light patterns in the first image and the second structured light patterns in the second image to obtain three-dimensional spatial position information of the to-be-measured object;
executing a color mixing process on portions of the first image and the second image corresponding to the second wavelength, a portion of the third image corresponding to the first wavelength, and a portion of the fourth image corresponding to the third wavelength, to obtain spatial color information of the to-be-measured object; and
combining the three-dimensional spatial position information and the spatial color information based on a spatial correspondence between the three-dimensional spatial position information and the spatial color information, so as to generate a target color three-dimensional model of the to-be-measured object.

13. The system according to claim 12, wherein, when the to-be-measured object is simultaneously illuminated with the first structured light and the second background light, a plurality of first background light patterns are determined based on the plurality of first structured light patterns, wherein the plurality of first structured light patterns and the plurality of first background light patterns are alternately arranged at a plurality of encoding positions to form a first encoding pattern, and when the to-be-measured object is simultaneously illuminated with the second structured light and the second background light, a plurality of second background light patterns are determined based on the plurality of second structured light patterns, and the plurality of second structured light patterns and the plurality of second background light patterns are alternately arranged at the plurality of encoding positions to form a second encoding pattern.

14. The system according to claim 13, wherein the encoding positions of the plurality of first background light patterns are complementary to the encoding positions of the plurality of second background light patterns.

15. The system according to claim 13, wherein: the plurality of first structured light patterns are respectively disposed at a plurality of odd-numbered positions among the plurality of encoding positions, the plurality of first background light patterns are respectively disposed at a plurality of even-numbered positions among the plurality of encoding positions, the plurality of second structured light patterns are respectively disposed at the plurality of even-numbered positions, and the plurality of second background light patterns are respectively disposed at the plurality of odd-numbered positions.

16. A system for establishing a color three-dimensional model, the method comprising:
a projection illumination module configured to illuminate a to-be-measured object with a first structured light, a second structured light, a third structured light, a fourth structured light, and a first background light of a first wavelength, a second background light of a second wavelength, and a third background light of a third wavelength, wherein the first structured light includes a plurality of first structured light patterns, the second structured light includes a plurality of second structured light patterns, the third structured light includes a plurality of third structured light patterns, the fourth structured light includes a plurality of fourth structured light patterns, and the plurality of first structured light patterns, the plurality of second structured light patterns, the plurality of third structured light patterns, and the plurality of fourth structured light patterns have different spatial characteristics;
an image sensing device configured to capture a first image when the to-be-measured object is simultaneously illuminated by the first structured light and the second background light, capture a second image when the to-be-measured object is simultaneously illuminated by the second structured light and the second background light, capture a third image when the to-be-measured object is illuminated by the first background light, capture a fourth image when the to-be-measured object is simultaneously illuminated by the third structured light and the third background light, and capture a fifth image when the to-be-measured object is simultaneously illuminated by the fourth structured light and the third background light;
a processing device configured to perform the following processes:
obtaining the first image, the second image, the third image, the fourth image, and the fifth image;
executing a decoding process on the plurality of first structured light patterns in the first image, the plurality of second structured light patterns in the second image, the plurality of third structured light patterns in the fourth image, and the plurality of fourth structured light patterns in the fifth image, so as to obtain three-dimensional spatial position information of the to-be-measured object;
executing a color mixing process on portions of the first image and the second image corresponding to the second wavelength, a portion of the third image corresponding to the first wavelength, and portions of the fourth image and the fifth image corresponding to the third wavelength, so as to obtain spatial color information of the to-be-measured object; and
combining the three-dimensional spatial position information and the spatial color information based on a spatial correspondence between the three-dimensional spatial position information and the spatial color information, so as to generate a target color three-dimensional model of the to-be-measured object.

17. The system according to claim 16, wherein the projection illumination module is further configured to illuminate the to-be-measured object with a fifth structured light of the first wavelength; and
the image sensing device is further configured to capture a sixth image when the to-be-measured object is illuminated by the fifth structured light.

18. The system according to claim 17, wherein the process of obtaining the three-dimensional spatial position information of the to-be-measured object further includes: executing the decoding process on a plurality of fifth structured light patterns of the fifth structured light.

19. The system according to claim 16, wherein, when the to-be-measured object is simultaneously illuminated with the third structured light and the third background light, the processing device is configured to determine a plurality of third background light patterns based on the plurality of third structured light patterns, and the plurality of third structured light patterns and the plurality of third background light patterns are alternately arranged at a plurality of encoding positions to form a third encoding pattern; and
when the to-be-measured object is simultaneously illuminated with the fourth structured light and the third background light, a plurality of fourth background light patterns are determined based on the plurality of fourth structured light patterns, wherein the plurality of fourth structured light patterns and the plurality of fourth background light patterns are alternately arranged at the plurality of encoding positions to form a fourth encoding pattern.

20. The system according to claim 12, wherein the processing device is further configured to perform the following processes:
executing a position detection process on the first image and the second image to obtain first pattern position information of the plurality of first structured light patterns in the first image and second pattern position information of the plurality of second structured light patterns in the second image; and
obtaining, according to the first pattern position information and the second pattern position information, color position information of the portions corresponding to the second wavelength in the first image and the second image, the portion corresponding to the first wavelength in the third image, and the portion corresponding to the third wavelength in the fourth image, and obtaining the spatial correspondence between the three-dimensional spatial position information and the spatial color information from the color position information.
